(19) United States
(12) Patent Application Publication
(10) Pub. No.: US 2024/0299021 A1
(43) Pub. Date: Sep. 12, 2024

(54) ILLUMINATED MAGNET SAFE RETRACTORS AND OTHER MEDICAL DEVICES

(71) Applicant: OBP Surgical Corporation, Lawrence, MA (US)

(72) Inventors: James PEHL, Salem, NH (US); Jason SWIFT, Newburyport, MA (US); Matthew TRAUB, Andover, MA (US)

(21) Appl. No.: 18/598,030

(22) Filed: Mar. 7, 2024

Related U.S. Application Data
(60) Provisional application No. 63/489,003, filed on Mar. 8, 2023.

(51) Int. Cl. A61B 17/02; A61B 17/00; A61B 90/35

(52) U.S. Cl. CPC A61B 17/02; A61B 90/35; A61B 2017/00831

(57) ABSTRACT

An illuminated surgical retractor including an outer housing forming a handle and a blade extending from the handle, an illumination assembly comprising at least one direct light source, wherein the outer housing houses therein one or more ferromagnetic components, and wherein the outer housing includes an exterior surface and an inner surface and wherein predetermined portions of one or more of the exterior surface and the inner surface of the outer housing have a shielding material applied thereto to prevent interference of the one or more ferromagnetic components housed within the outer housing with magnetic and electromagnetic signals.

### RELATED APPLICATIONS

The present application claims priority to U.S. Provisional Application No. 63/489,003 filed on Mar. 8, 2023. The entire disclosure of this provisional application is incorporated herein by reference.

### FIELD OF INVENTION

The present invention relates to medical devices and, more particularly, to illuminated medical devices having light sources to illuminate surgical fields and being configured to be magnet safe so as to prevent interference with external magnetic fields.

### BACKGROUND

Illuminated medical devices, such as illuminated retractors and illuminated suction devices, are used in the medical field for illuminating body cavities during surgical procedures. Examples of illuminated retractors, illuminated suction devices and other medical devices are disclosed in U.S. Pat. Nos. 10,512,519; 10,799,229; 10,966, 702; 10,959,609; 10,420,540; 11,197,662; 10,939,899; 10,912,455; 10,441, 155; 10,881,387; 10,722,621; 10,420,538; 11,439,379 and 10,966,699; and U.S. application Ser. Nos. 17/014,385; 17/192,935; 17/212,506 and 17/171,423, assigned to the same Assignee herein, disclosures of which are incorporated herein by reference.

In order to provide illumination, such medical devices include electrical components that use or incorporate ferromagnetic materials, such as cobalt, iron and nickel, as part of an illumination assembly and in some cases, in other portions of the device. As a result, illuminated medical devices would not be suitable or appropriate for use with other medical equipment that uses and/or relies on magnetic waves due to interference of the illuminated medical device with magnetic waves or magnetic fields. Specifically, if a conventional illuminated medical device is used when an external magnetic field is applied during the medical/surgical procedure, such medical device would cause distortions and disruptions in the magnetic fields, thus reducing the accuracy of the magnetic devices or magnetic resonance (MR) devices. In addition, externally applied magnetic fields may cause heating of illuminated medical device components, thus requiring additional heat sinking or heat elimination from the illuminated medical device.

### SUMMARY OF THE INVENTION

The present invention provides an illuminated medical device which is magnet safe—namely, the illuminated medical device of the present invention can be safely used with externally applied magnetic and electromagnetic fields, contemporaneously with magnetic detection equipment and/or with magnetic resonance equipment, without affecting, distorting or interfering with magnetic/electromagnetic fields and magnetic/electromagnetic signals. In addition, the illuminated medical device of the present invention does not require additional heat sinking when used with magnetic detection or magnetic resonance equipment. That is, the illuminated medical device can be safely used while magnetic and electromagnetic fields are applied, within the application field or area of the magnetic and electromagnetic fields, without distorting or interfering with the magnetic or electromagnetic fields and signals and without requiring additional heat sinking.

In accordance with the invention, the illuminated surgical retractor comprises an outer housing forming a handle and a blade extending from the handle, an illumination assembly comprising at least one direct light source, wherein the outer housing houses therein one or more ferromagnetic components, and wherein the outer housing includes an exterior surface and an inner surface and wherein predetermined portions of one or more of the exterior surface and the inner surface of the outer housing have a shielding material applied thereto to prevent interference of the one or more ferromagnetic components housed within the outer housing with magnetic and electromagnetic signals. In certain embodiments, the shielding material is one or more of paramagnetic material, diamagnetic material and non-magnetic material capable of shielding against magnetic and electromagnetic field interference. An exemplary shielding material is one or more of copper and aluminum. In some embodiments, the shielding material has a minimum thickness of 0.001 mm, and in some embodiments, the thickness range is 0.001-0.51 mm.

In certain embodiments of the surgical retractor, the predetermined portions of the one or more of the exterior surface and the inner surface of the outer housing include a joint between the handle and the blade, a proximal portion of the blade and a proximal portion of the handle. In an exemplary embodiment, the proximal portion of the blade extends at least 1 inch from the joint between the handle and the blade, and the proximal portion of the handle extends at least 1 inch from the joint between the handle and the blade.

In certain embodiments, the outer housing of the retractor comprises a polymer material and wherein the shielding material is applied to the predetermined portions of the one or more of the exterior surface and the inner surface of the outer housing using electroplating.

In some embodiments, the illumination assembly comprises a flexible circuit and the at least one direct light source is mounted on the flexible circuit, the flexible circuit is housed within the blade and includes a flexible circuit board and copper layer provided on a top and a bottom surface of the flexible circuit board, and the shielding material is not required to be applied to portions of the blade housing the flexible circuit board with the copper layer thereon.

In certain embodiments, the one or more ferromagnetic components include one or more ferromagnetic fastening members, one or more power sources housed in the handle and a control assembly housed in the handle for controlling power supply to the at least one direct light source. In some embodiments, at least one of the one or more ferromagnetic components includes shielding material directly applied thereto.

In accordance with the present invention, a medical device comprises an outer housing forming a handle and an operative portion extending from the handle, one or more ferromagnetic components housed within the outer housing, wherein the outer housing includes an exterior surface and an inner surface, and wherein predetermined portions of one or more of the exterior surface and the inner surface of the outer housing have a shielding material applied thereto to prevent interference of the one or more ferromagnetic components housed within the outer housing with magnetic and electromagnetic signals.

In some embodiments, the shielding material is one or more of paramagnetic material, diamagnetic material and non-magnetic material capable of shielding against magnetic and electromagnetic field interference. Exemplary shielding material is one or more of copper and aluminum. In certain embodiments, the shielding material has a minimum thickness of 0.001 mm and a thickness range of 0.001-0.51 mm.

In the medical device of the present invention, the predetermined portions of the one or more of the exterior surface and the inner surface of the outer housing include portions of the outer housing enclosing the one or more ferromagnetic components and located inside a surgical pocket or within 0-4 inches of a surgical pocket opening when the medical device is in use. In certain embodiments, the predetermined portions of the one or more of the exterior surface and the inner surface of the outer housing include at least a portion of the operative portion, a proximal portion of the handle and a joint between the handle and the operative portion.

In some embodiments, the outer housing comprises a polymer material and wherein the shielding material is applied to the predetermined portions of the one or more of the exterior surface and the inner surface of the outer housing using electroplating.

In certain embodiments, the one or more ferromagnetic components include one or more ferromagnetic fastening members and one or more power sources. At least one of the one or more ferromagnetic components may include shielding material applied thereto.

In some embodiments of the present invention, the medical device is one of a retractor, a dual blade retractor, a suction device, a speculum, an anoscope, an electrocautery device and a laryngoscope.

The present invention further includes an illuminated surgical retractor comprising an outer housing forming a handle and a blade extending from the handle, an illumination assembly comprising at least one direct light source and one or more power sources, wherein the outer housing is formed from a polymer material and houses therein one or more ferromagnetic components, and wherein shielding material is selectively applied to one or more of predetermined portions of the outer housing and the one or more ferromagnetic components to prevent interference of the one or more ferromagnetic components with magnetic and electromagnetic signals. The shielding material may be one or more of copper and aluminum. In some embodiments, the shielding material has a minimum thickness of 0.001 mm, and a thickness range of 0.001-0.51 mm.

### BRIEF DESCRIPTION OF THE DRAWINGS

The above and other features and aspects of the present invention will become more apparent upon reading the following detailed description in conjunction with the accompanying drawings, in which:

FIGS. 1A-1C show views of a retractor with multiple light sources in accordance with a first embodiment of the present invention;

FIG. 2 shows a cutaway view of the retractor of FIGS. 1A-C;

FIG. 3 shows a retractor in accordance with a second embodiment of the present invention;

FIGS. 4A-4D show various views of the retractor of FIG. 3 including a smoke evacuation system and an LED flex circuit;

FIG. 5A shows an LED flex circuit of the retractor of FIGS. 4A-D;

FIGS. 5B-5C show a modified LED flex circuit with improved thermal dissipation;

FIG. 5D shows a table with exemplary characteristics of various layers of the LED flex circuit of FIGS. 5A-C;

FIGS. 6A-6D show a retractor in accordance with another embodiment of the present invention;

FIGS. 7A and 7B show exploded views of a retractor of FIGS. 3 and 6A-6D, respectively;

FIGS. 7C-7D show an internal view of the handle portion of the retractor of FIGS. 7A-B in an assembled state;

FIG. 8 shows power connections among different components of the retractor of FIGS. 7A-B;

FIGS. 9A and 9B illustrate retractors of FIGS. 1-7 with annotations to indicate portions of the retractor that are likely to interfere with external magnetic equipment;

FIG. 10 is an enlarged view of a portion of the retractor of FIG. 9B that is most likely to be within or near a surgical pocket;

FIG. 11 shows an electroless, auto-catalytic, chemical plating process for electroplating the shielding material on the predetermined portions of the polymer housing; and

FIG. 12 shows another process for electroplating the shielding material on the polymer housing.

### DETAILED DESCRIPTION

It is noted that embodiments described herein relate to various features of an illuminated medical device, such as a surgical retractor. While some of these features are described from the perspective of being embodied within a retractor, such illustration is not intended to limit the scope of the present invention. Other medical devices such as a dual-blade retractor, a vaginal speculum, a laryngoscope, an anoscope, a suction device, an electrocautery device, etc. may also fully embody the features described herein. For example, a dual-blade retractor or a vaginal speculum may embody the features of the present invention relating to an illumination assembly having multiple light sources, such as LEDs, that are particularly configured to maximize illumination while minimizing obstruction of the physician's field of view. As another example, an illuminated laryngoscope, an illuminated suction device or an illuminated electrocautery device may incorporate the features of the present invention relating to removal of batteries and disposability of the device. The illuminated medical devices, including retractors, dual-blade retractors, speculums, suction devices, etc., also incorporate magnetically shielded features described herein below to make these devices magnet safe. These examples are not intended to limit the full scope of the present invention, and the various medical devices may include one or more of the features described herein below.

The illuminated medical device of the present invention includes multiple features, which may be used in different devices and in different versions of the medical devices. These features include application or incorporation of shielding to certain portions of the medical device to prevent or minimize interference with magnetic fields and signals. Other features of medical devices include use of multiple LEDs for illumination and control thereof, a smoke evacuation system integrated with an LED flex circuit for illumination, and a modified LED flex circuit for improved thermal dissipation. These features are described with reference to particular embodiments of a surgical retractor shown in the drawings. However, these features may be incorporated in each of the embodiments or variations of the retractor or may be selectively incorporated in the different variations of the retractor. Moreover, as mentioned herein above, these features may be used in other types of medical devices. The features of the illuminated medical devices will now be described in detail.

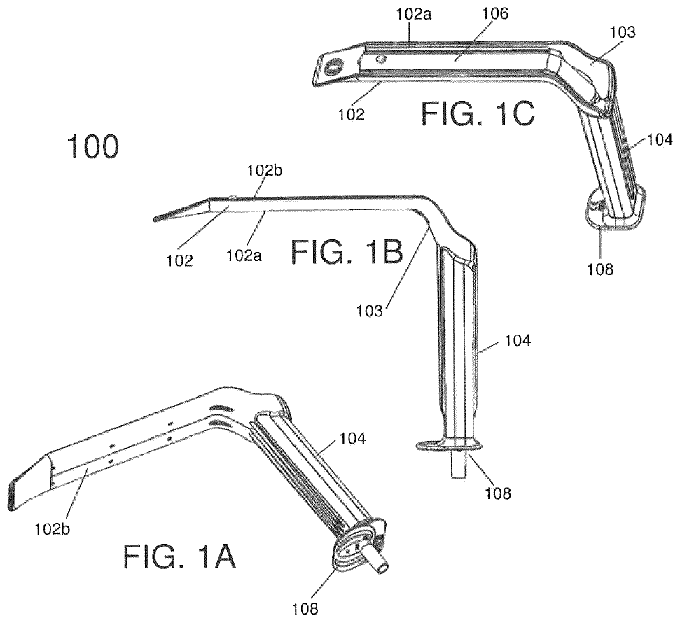

Referring to the drawings, FIGS. 1A-1C thereof show different views of a retractor 100 in accordance with a first embodiment of the present invention. As shown in these figures, the retractor 100 includes a blade portion 102 having a first (bottom) surface 102a and an opposing second (bottom) surface 102b and a proximal end and a distal end. The proximal end is defined herein as the end of the blade portion closer to a handle of the retractor and the distal end is defined as the tip end of the blade portion opposing the proximal end. The retractor 100 further includes a handle portion 104 that extends from the blade portion 102 at an angle. In this illustrative embodiment, the handle portion 104 is generally perpendicularly joined to the blade portion at the proximal end of the blade portion. In some versions, the angle may vary, for example, at 95°, 100°, 105°, etc., without limiting the scope of the present embodiment. The portion of the retractor 100 where the blade portion 102 and the handle portion 104 are joined is referred to herein as a curved portion 103 or a saddle portion 103.

The retractor 100 further includes a smoke evacuation channel formed by a cover 106 that runs along the length of the blade portion 102 to the handle portion 104 and may be in fluid communication with an end cap assembly 108 at a distal end of the handle portion 104. The end cap assembly 108 in this embodiment is insertable into an open distal end of the handle portion 104. However, in other embodiments, the end cap assembly 108 may be integrally formed with the distal end of the handle portion 104 or may be attached to the distal end of the handle portion in a different manner. The cover 106 may have an arc shape, semi-circular shape, rectangular shape, or other suitable shape or size appropriate for carrying out the function of evacuating smoke or debris generated near the distal end of the retractor 100 to the end cap assembly 108. In the version shown in FIGS. 1A-C, the end cap assembly 108 is provided with a vacuum port which can be attached to a vacuum source and is in fluid communication with the smoke evacuation channel. In other variations, the vacuum port may be provided in other areas of the handle portion, such as near the proximal end of the handle portion, instead of in the end cap assembly. The smoke evacuation channel serves as an air conduit to direct smoke, fumes and/or debris from the distal end of the blade portion, through the length of the retractor, and out the end cap assembly 108 at the bottom of the handle portion 104. As mentioned above, the end cap assembly 108 can be coupled with a vacuum source to suck the smoke, fumes and/or debris via the smoke evacuation channel.

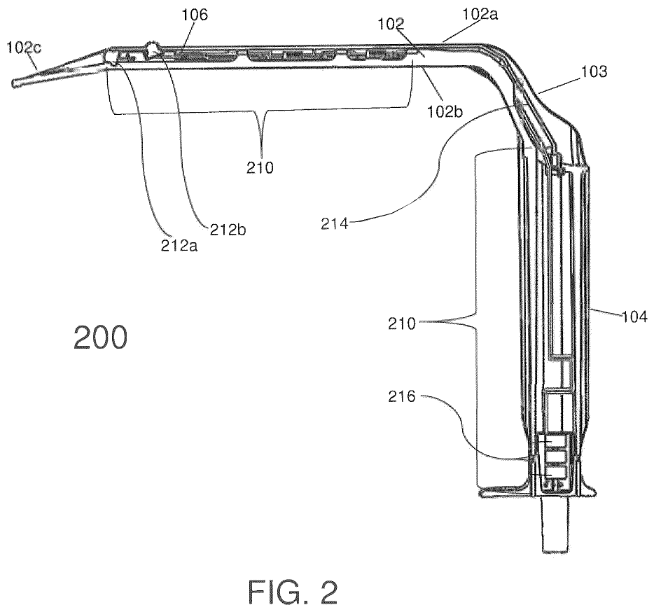

As shown in FIG. 1C, the smoke evacuation channel cover 106 is coupled to the top surface 102a of the blade portion 102 and extends at an angle along the surface of the saddle portion 103. As shown in FIG. 2, the handle portion 104 has a hollow interior. In some illustrative versions, the smoke evacuation channel cover 106 opens at one end into an opening formed at the proximal end of the handle portion. In other illustrative versions, the smoke evacuation channel cover 106 may be coupled with the handle portion 104 or may extend into the opening formed at the end of the handle portion 104. In yet other versions, the handle portion 104 may include an internal channel in fluid communication with the smoke evacuation channel cover 106. In certain versions, the smoke evacuation channel cover 106 is permanently attached to at least the blade portion. The attachment between the cover 106 and the blade portion 102 may be achieved by any suitable means. In one illustrative version, the cover 106 is provided with engagement tabs and the blade portion includes corresponding slots, visible in FIG. 1A, wherein the engagement tabs on the cover 106 are received by and engaged with the corresponding slots in the blade portion. A more detailed description of the engagement tabs and corresponding slots is provided in U.S. Pat. No. 9,867,602 and in U.S. Published Application No. 2017/0245849, both assigned to the same assignee herein, and incorporated herein by reference.

Additional details as to the structural aspects of the blade portion, the handle portion, and the smoke evacuation channel can be found in U.S. Published Application No. 2016/0354072, titled “RETRACTOR”, and in U.S. Pat. No. 10,512,519, titled “ILLUMINATED MEDICAL DEVICES,” the entire contents of which is incorporated herein by reference. Therefore, additional descriptions of these elements will be omitted for sake of brevity.

FIG. 2 shows a cutaway view of a retractor 200 in accordance with the first embodiment of the present invention. In particular, the retractor 200 includes an illumination assembly 210 that extends along the lengths of the blade portion and also the handle portion. More specifically, the illumination assembly 210 includes at least a first light source 212a, a second light source 212b, a power source, such as batteries 216, and wires or connections 214 coupling the light sources 212a, 212b with the batteries 216. The first and second light sources 212a and 212b are connected to the batteries 216 via the wires 214 which run along the length of the retractor. Although not shown, the illumination assembly 210 may further include one or more switches or activation devices to selectively control energization of the light sources. The activation device may be a depressible switch, a rotatable switch, a flickable switch, a slidable switch, a removable pull tab, a touch button, a lever, a multi-directional switch, a motion detection assembly, etc or a combination thereof.

The positioning of the batteries 216 as shown in FIG. 2 is also not intended to be limiting. In fact, the positioning of the batteries, and therefore, the configuration of the one or more wires to connect the light sources to the batteries may vary without departing from the scope of the present invention. For example, the batteries may be disposed adjacent to the saddle portion 103, on or at least partly on the saddle portion 103, or may be disposed at a different position within the handle portion 104. As another example, the batteries, or at least a portion of the batteries, may be contained within a battery compartment that is placed externally, or at least partly externally, to the handle portion 104 and otherwise attached to an external surface of the handle portion 104 or at another location on the retractor 200. In yet another example, the batteries may be provided on or attached to the blade portion of the retractor, on either the same surface of the blade as the smoke evacuation cover 106 or the opposing surface of the blade.

FIG. 2 illustrates a specific configuration of the first light source 212a and the second light source 212b, and the other portions of the illumination assembly, which is integrated with the smoke evacuation channel and its cover 106. The first light source 212a is provided at an opening formed at the end of the smoke evacuation channel cover 106, and the first light source 212a is partially enclosed by the end of the smoke evacuation channel cover 106. The second light source 212b is provided at a distance away from the first light source 212a closer to the proximal end of the blade portion. The second light source 212b extends and/or protrudes through an opening formed in the smoke evacuation channel cover 106. The second light source 212b is also angled relative to the blade portion 102 at a second angle which is different from the first angle, so that the light emitted by the second light source 212b is directed at a different angle and more toward an area outside the first surface 102a of the blade 102. In some versions, the first light source 212a may be at an angle between 0° and 30° with respect to the horizontal plane of the main portion of the retractor blade, and the second light source 212b may be at an angle between 60° and 90° with respect to the same horizontal plane. More detailed descriptions of the retractor of FIGS. 1A-2, variations thereof, the details of the illumination assembly in the retractor of FIGS. 1A-2 and control of the light sources 212 of the illumination assembly are described in U.S. Pat. No. 10,512,519, assigned to the same assignee herein, the subject matter of which is incorporated herein by reference. Therefore, additional descriptions of these features will be omitted for the sake of brevity.

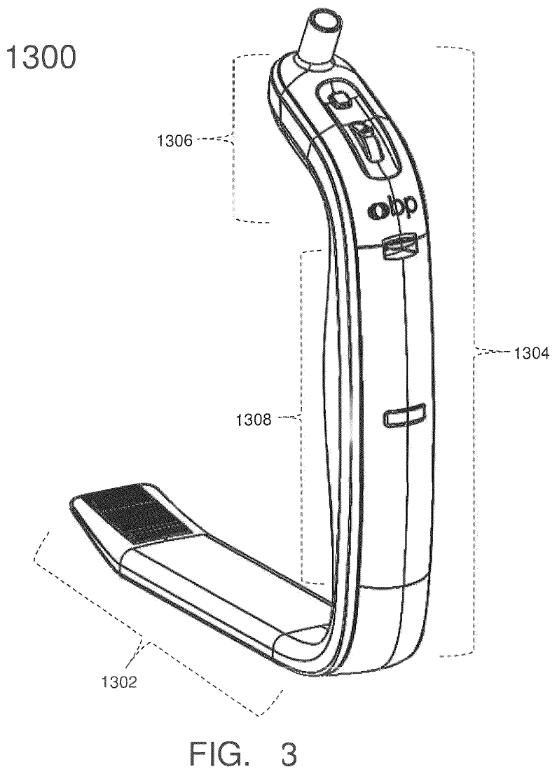

FIG. 3 shows a retractor 1300 in accordance with a second embodiment of the present invention. As shown in FIG. 3, the retractor 1300 includes a blade portion 1302 and a handle portion 1304. Unlike the retractor of the first embodiment, the retractor 13 in FIG. 3 does not include side flanges extending outwardly from a curved section connecting the blade portion 1302 and the handle portion 1304. This feature allows for better viewing of the surgical site and results from the construction of the blade 1302 and a blade cover, as described in more detail below. Moreover, the retractor 1300 of this embodiment does not include an end cap assembly which is insertable into an opening at the distal end of the handle portion. Instead, a distal end portion of the handle portion 1304 of the retractor 1300, where an end cap assembly would have been configured, extends into a curved end portion 1306 which is offset from the plane along the length of the handle portion 1304.

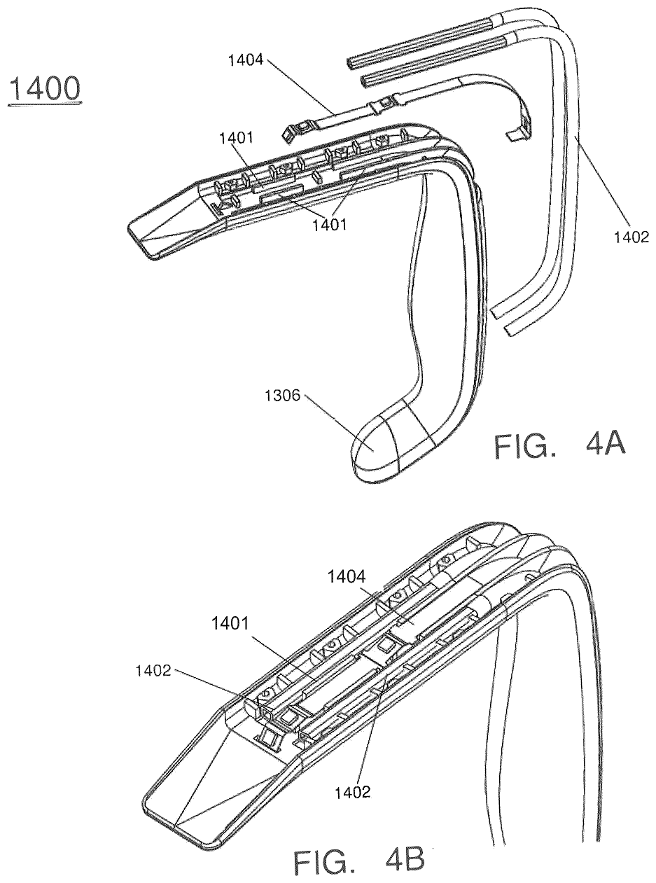

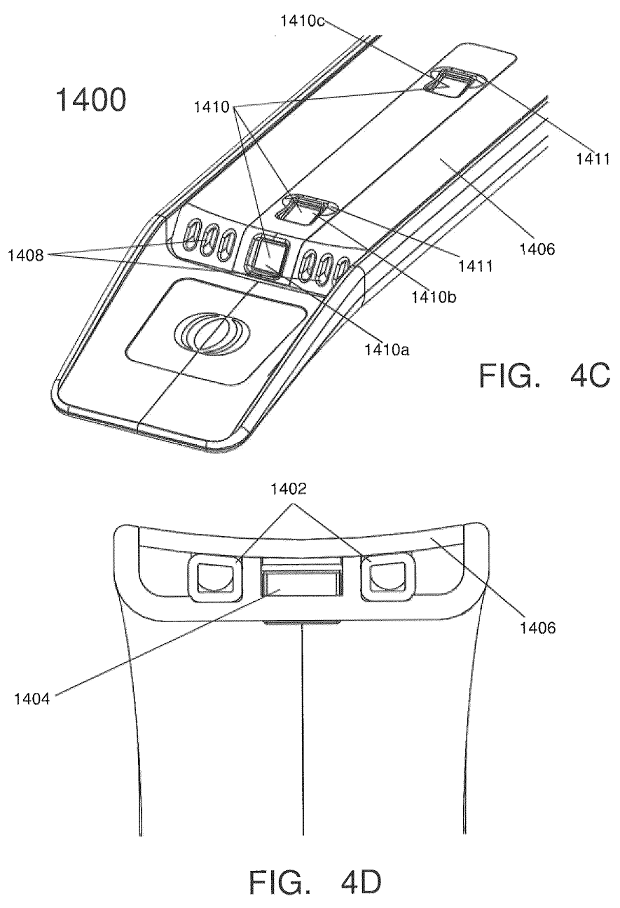

The curved end portion 1306, similar to an end cap assembly as described above herein, includes a smoke evacuation port along with an operating assembly to control operation of one or more LEDs of the retractor and/or other retractor functionalities. In accordance with certain versions of the present embodiment, the curved end portion 1306 may be referred to as being part of the handle portion 1304, while in some other versions, the curved end portion 1306 may be referred to as a separate component that is adjacent to, or otherwise attached to, the handle portion 1304 of the retractor 1300. Specifics of the purpose, functionalities, and/or components of the curved end portion 1306 are described in detail in U.S. Pat. No. 10,512,519 incorporated herein by reference. In other variations, the distal end portion 1306 is not curved and may be co-extensive with the remainder of the handle or may have any other suitable configuration. Finally, the handle portion 1304 of the retractor 1300 is configured with a power source storage portion 1308 that stores a power source, such as one or more batteries, and includes a push-tab assembly described in detail in U.S. Pat. No. 10,512,519 incorporated herein by reference. FIGS. 4A-4D show various views (e.g., a perspective exploded view, enlarged views and a cross-sectional view) of the retractor 1400 which is substantially the same as or similar to the retractor 1300 described with reference to FIG. 3. The exploded view of the retractor 1400 in FIG. 4A shows that the retractor 1400 includes a pair of tubes (or conduits) 1402 and an LED flex circuit 1404. In accordance with this embodiment, the pair of tubes 1402 function as and/or replace the smoke evacuation channel described herein above with reference to the previous embodiments. Similar to the smoke evacuation channel in retractors of the previous embodiments, the pair of tubes 1402 run along the blade portion and the handle portion of the retractor 1400 and are in fluid communication with the smoke evacuation port positioned on the curved end portion of the retractor 1400. More specifically, the pair of tubes 1402 serve as fluid conduits to direct smoke, fumes, liquids and/or small debris from the distal end of the blade portion, through the length of the retractor 1400, and out the smoke evacuation port located at the curved end portion of the retractor 1400. While in the illustrative version of FIGS. 4A-4D, two tubes 1402 are provided for smoke and/or debris evacuation, in other versions, only one tube may be provided or more than two tubes may be used.

In accordance with various embodiments disclosed herein, the medical devices of the present invention include an illumination assembly, which comprises one or more light sources (e.g., LEDs), and the illumination assembly is integrated into the blade portion of the retractor along with a blade cover. The blade cover provides the air-conduit space and functions as a smoke evacuation channel. In the previous embodiment, the positions of the LEDs of the illumination assembly correspond with the positions of a plurality of openings formed in the blade cover to allow light to escape via the plurality of openings.

In accordance with the present embodiment, the retractor 1400 has an illumination assembly comprising the LED flex circuit 1404 that functions as and/or replaces the one or more LEDs and wiring of the illumination assembly described herein above. The LED flex circuit 1404 includes a flexible substrate, in this version formed as a flexible strip, with one or more LEDs mounted thereon. The LED flex circuit 1404 incorporates wiring and electrical connections in the flexible substrate for electrically connecting the LED(s) to the power source and to an operating assembly and/or a control assembly for controlling the LED(s). The flexible substrate may also incorporate therein one or more circuits for controlling the operation of the LEDs. The LED flex circuit 1404 also includes a connecting portion to provide an easy electrical connection to other components of the illumination assembly, e.g., the power source and/or the operating assembly.

Moreover, the retractor 1400 includes the pair of tubes 1402, each of which run separately and adjacent to the LED flex circuit 1404 on either side thereof. As discussed above, the pair of tubes 1402 extend from the blade portion into the handle portion and extend into the distal end portion 1306 of the handle. In some versions, the tubes 1402 end in the distal end portion 1306 near the smoke evacuation port, while in other versions, the tubes 1402 may be coupled to the smoke evacuation port in the distal end portion 1306. In yet other variations, the tubes 1402 may terminate at another point within the handle portion of the retractor.

Additionally, the retractor 1400 includes a blade cover 1406, a portion of which is shown in FIG. 4C, that encloses both the LED flex circuit 1404 and the pair of tubes 1402. In some versions, the blade cover 1406 covers the entire width of the blade portion of the retractor 1400, while in other versions, the blade cover 1406 covers only a portion of the width, e.g., the width of the LED flex circuit 1404 and the pair of tubes 1402. The length of the blade cover 1106 may be similar to the smoke evacuation channel of the previous embodiments. In FIG. 4C, a distal end of the blade cover 1406 covers distal ends of the pair of tubes 1402 and the LED flex circuit 1404, and is positioned at or near an angled tip of the blade portion. A proximal end of the blade cover 1406 may end at the proximal end of the blade or may extend to cover a curved portion connecting the blade to the handle and even to cover a proximal end portion of the handle portion.

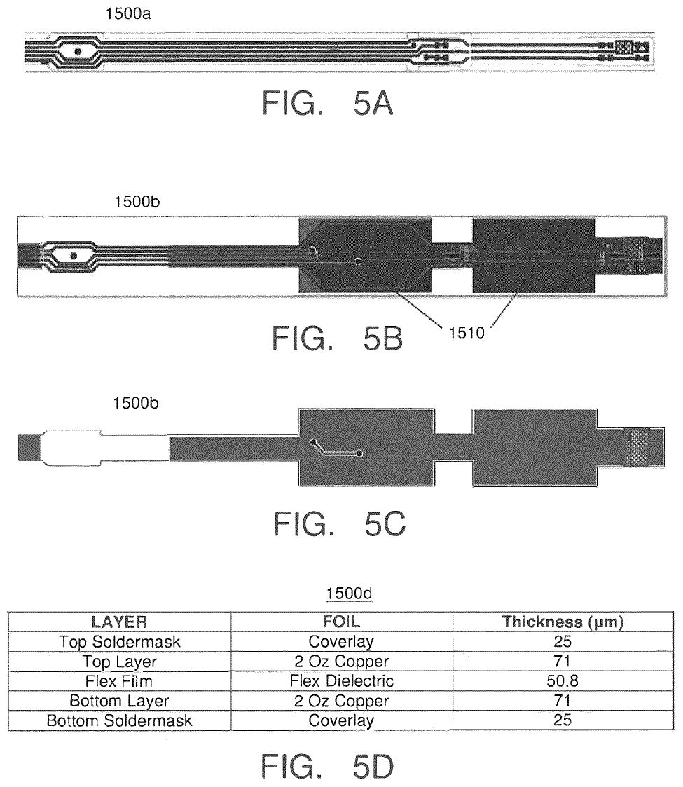

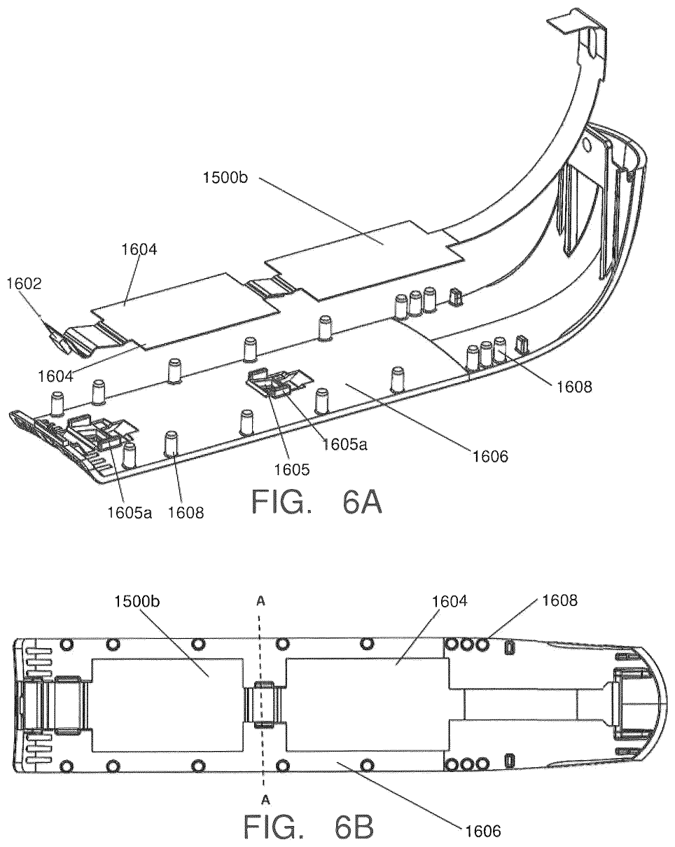

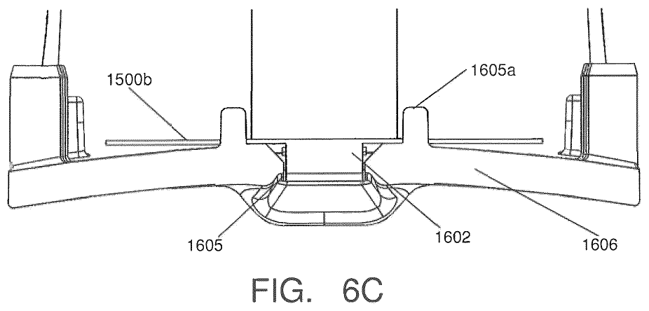

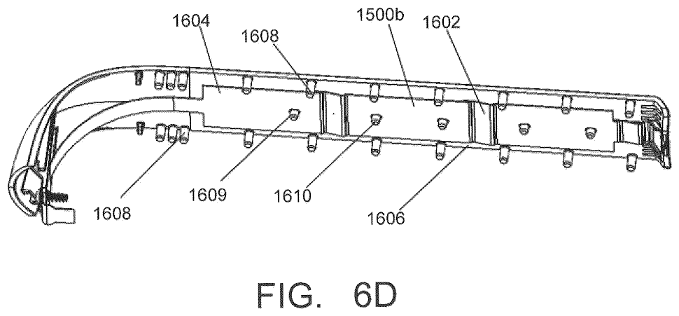

FIG. 4B illustrates the retractor 1400 in which the pair of tubes 1402 and the LED flex circuit 1404 are assembled in their respective positions on the blade portion of the retractor 1400, without the blade cover 1406. The LED flex circuit 1404 enables the positions and/or the angles of the LEDs to be individually defined by the support features of the blade, e.g., support ribs or projections on the blade, and the blade cover 1406. Moreover, as shown in FIG. 4B, the LED flex circuit 1404 may be retained between a plurality of retaining walls 1401, which provide additional support for the LED flex circuit 1404 and additional strength to the blade. In the illustrative version of FIG. 4B, the retaining walls 1401 are provided at selected positions to retain selected portions of the LED flex circuit 1404 and so as not to abut the LEDs mounted on the LED flex circuit 1404. This configuration allows for easy adjustment of the angles of the LEDs. However, in other versions, the retaining walls may be provided in other positions, including those abutting the LEDs of the LED flex circuit 1404. In this embodiment, the retaining walls 1401 also separate the LED flex circuit 1404 from the tubes 1402 and help retain the tubes 1402 in their respective positions, so as to prevent shifting of the tubes 1402 and of the LED flex circuit 1404 and overlapping between portions of the tubes 1402 and the LED flex circuit 1404. In other embodiments, the retaining walls may be omitted or limited and the LED flex circuit may be modified, as shown in FIGS. 5B-C and 6A-D, to include wings which overlap with the tubes of the smoke evacuation system. In some embodiments using the LED flex circuit shown in FIGS. 5B-C and 6A-D, the retaining walls may be used only for retaining the tubes 1402 in place and the LED flex circuit may be positioned adjacent to the blade cover 1606 as shown in FIG. 6B and when assembled with the blade, the LED flex circuit wings cover and overlap with the tubes of the smoke evacuation system.

FIG. 4C shows a distal end portion of the blade cover 1406 that covers or encloses the upper surface, or a portion of the upper surface, of the retractor blade portion to enclose the pair of tubes 1402 and the LED flex circuit 1404. In the version shown in FIG. 4C, the cover has a top surface, which is substantially parallel to the upper surface of the blade portion, and a distal end extending at an angle from the top surface toward the upper surface of the blade portion. FIG. 4C shows that the blade cover 1406 includes a pair of gratings or grills 1408 each of which corresponds to, and aligns with, a respective tube 1402. The gratings 1408 are provided in the distal end of the cover 1106 and serve as a filter to block out larger debris that might clog the tubes 1402 during fluid and/or smoke evacuation processes. The particular sizes, shapes and designs of the gratings 1408 may be varied without departing from the scope of the present invention. For example, the size of the grate openings may depend on the type of surgery for which the retractor is used.

As further shown in FIG. 4C, the blade cover 1406 of the retractor 1400 also includes a plurality of openings 1410, with each of the openings corresponding to a respective LED mounted on the LED flex circuit 1404. In the version of FIG. 4C, the plurality of openings include a first opening 1410a provided in the distal end of the cover 1406 between the gratings 1408, with a corresponding LED exposed through the first opening. The angle of the LED exposed through the first opening 1410a may be aligned with the angle of the distal end of the cover 1406 or may be provided at an angle relative to the distal end of the cover 1406. In addition, the angle of the LED exposed through the first opening may be adjustable.

In the version of FIG. 4C, a second opening 1410b is provided near the distal end of the cover 1406 in the top surface of the cover 1406 and a corresponding LED is exposed through the second opening 1410b. The LED exposed through the second opening 1410b may be angled relative to the top surface of the cover 1406 and its angle may be adjustable. In addition, the LED may be positioned such that a portion thereof protrudes from the second opening 1410b and/or may be positioned such that a portion thereof is below the top surface of the cover 1406. The cover may include one or more projections 1411 or ridges protruding from the surface of the cover 1406 and adjacent each opening on the top surface of the cover 1406, e.g., adjacent the openings 1410b, 1410c, in order to block light emitted from the LEDs from being directed toward the proximal end of the blade portion and/or toward the user of the retractor. In the embodiment of FIG. 4C, the projections 1411 extend adjacent a proximal end of each opening 1410b, 1410c on the top surface of the cover 1406. In some embodiments, the projections may extend slightly above the LED exposed through the opening 1410b, 1410c in order to block additional light emitted from the LED. In certain embodiments, the projection 1411 may have a reflective inner surface that faces the LED in order to direct light emitted from the LED toward the distal end of the blade portion. The plurality of openings 1410 may also include a third opening 1410c and/or other openings with corresponding LEDs exposed therethrough. In the version of FIG. 4C, the third opening 1410c has substantially the same or similar configuration to the second opening 1410b and the angle of the LED exposed through the third opening 1410c may be the same or different from the angle of the LED exposed through the second opening 1410c.

FIG. 4D is a detailed cross-sectional view of the blade portion of the retractor 1400 showing the pair of tubes 1402 running along each side of the LED flex circuit 1404 under the blade cover 1406 of the blade portion of the retractor 1400.

It is to be noted that the particular use of the LED flex circuit 1404 and the blade cover 1406 as shown in these figures is not intended to be limiting. In particular, the quantity, placement and positioning of LEDs on the LED flex circuit is subject to custom design, and thus, a corresponding blade cover may be appropriately designed to accommodate a particular design of LED flex circuit(s). In addition, the angles of the LEDs with respect to the blade portion may vary and be adjustable without departing from the scope of the present invention.

### Modified LED Flex Circuit for Improved Thermal Dissipation

As previously discussed herein, lighting of body cavities with an illuminated medical device comes with the well-documented problem of heat being created as a by-product and causing tissue damage. Even highly efficient LEDs only convert 30-40% of their energy into light and the rest is converted into heat.

The present invention recognizes that typical LEDs have thermal rises of 100-150 degrees C./Watt and that tissues exposed to 60° C. of heat even for a few seconds can be severely damaged. There is a thermal limit that bars the amount of power that can be fed to an LED. As a result, the present invention contemplates that power levels into an LED must be limited to approximately 150 mW or less to limit the thermal rise above the temperature of the surgical pocket to below 60° C.

The present embodiment focuses on the use of an LED flex circuit to accommodate various LED mounting angles, as well as for transmission of electrical signals across a surgical retractor. In further accordance with the present embodiment, various modifications to traditional flex circuits are contemplated in order to overcome the shortcomings of the existing art. First, in accordance with the present embodiment, the normal PCB copper thickness is doubled from 35 μm to at least 70 μm (i.e., at least 2 oz of copper thickness). This change in the copper thickness of the PCB halves the thermal resistance of the copper path. Second, the resistance path is changed by covering all available areas of the PCB substrate with copper. This is done by making electrical contact paths wide enough to cover the complete PCB substrate on both the top and the bottom surfaces, except for a minimum spacing required between electrical contact paths. This change maximizes the board's ability to conduct heat in all directions away from the LED. Third, the PCB substrate area is increased through the use of tabs or wings. Tabs added to the substrate are more than two times the normal width required for an LED printed circuit board. These tabs can be configured to fit in an unused area of the surgical instrument and thus not impede functionality. The combination of increased copper thickness, increased copper coverage on the top and bottom surfaces of the board, and increased board area collectively reduces the thermal resistance to 75° C. or less, and allows for twice as much power into the LEDs without sacrificing patient safety. Doubling the power into the LEDs will also double the amount of light, i.e., brightness, provided, thus significantly improving surgical pocket visualization while maintaining low cost.

Exemplary illustrations of the above-described modifications to the LED flex circuit are shown in FIGS. 5B-SC. FIG. 5A shows an LED flex circuit 1500a similar to the LED flex circuit shown in FIGS. 4A-B. FIG. 5B shows a top view of a modified LED flex circuit 1500b with improved copper coverage and improved board area through the use of wings 1510 outside of the LED area. FIG. 5C shows a bottom view (i.e., the underside) of the LED flex circuit 1500b showing additional copper coverage. As shown in these figures, the addition of wings in the LED flex circuit 1500b of FIGS. 5B-C, and other modifications to the LED flex circuit in accordance with the present embodiment maximize the amount of copper usage for a given application.

In the LED flex circuits 1500a-b of FIGS. SA-5C, a copper layer is provided on both sides of the LED flex circuit and the thickness of the copper layer is at least 2 oz or at least 70 μm, as described above. In addition to the copper layers provided on both sides of the LED flex circuit substrate, the LED flex circuits may also include a coverlay top layer, i.e., top soldermask, provided on one or both sides of the board as an additional electrically insulating layer. The electrically insulating coverlay passivates the LED flex circuit or portions thereof so that the passivated areas of the LED flex circuit can work with the smoke evacuation system described above to provide airflow over the LED flex circuit and thus, further decrease the thermal resistance from the LED to ambient. With such airflow over the LED flex circuit, additional thermal reductions of more than two times the previous reductions can be expected and thus, would allow even more power to be provided to the LEDs, resulting in higher luminous intensity.

FIG. 5D shows a Table 1500d that provides an exemplary set of values for various layers of the LED flex circuit board. As shown in Table 1500d, the thickness of the copper coverage has nearly doubled, both on the top layer and the bottom layer, to 2 oz of copper thickness, or 71 μm. As also shown in Table 1500d, the LED flex circuit of the present embodiment can include one or more layers of soldermasks, or coverlays, which are electrically insulating and are used to passivate the board. These top soldermasks have a thickness of about 25 μm. The specific values (or range of values) or the specific materials described are exemplary and are not intended to limit the scope of the present invention.

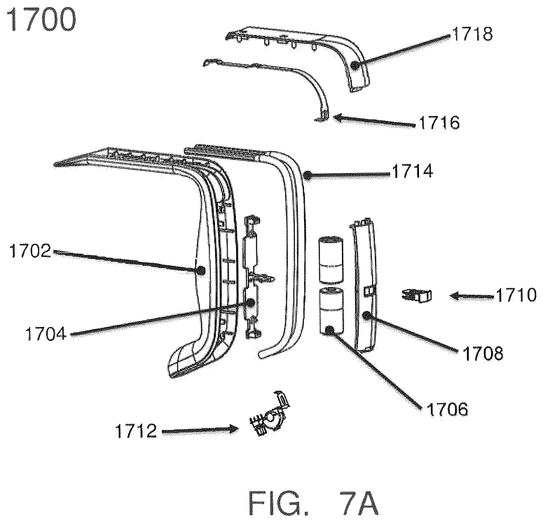

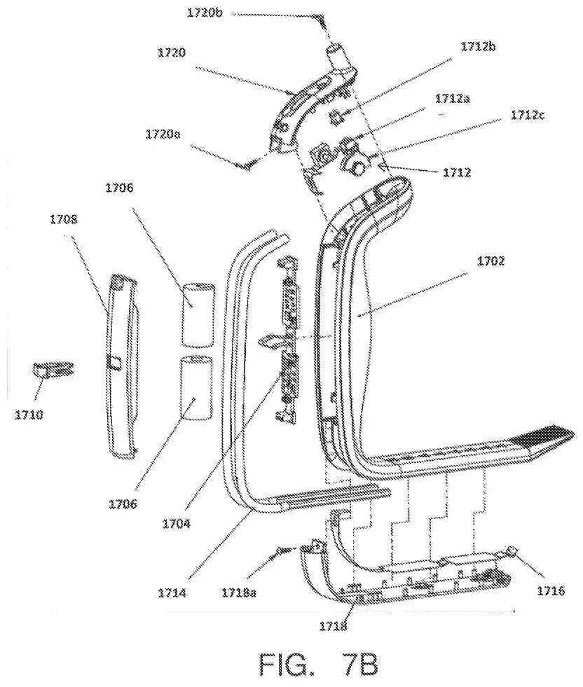

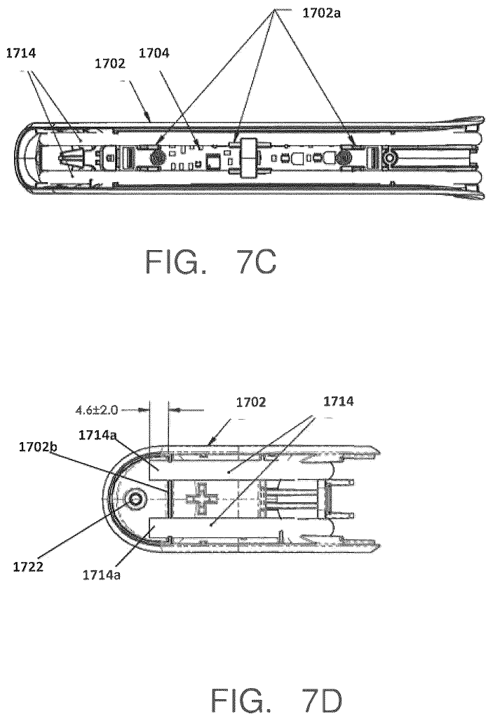

FIGS. 6A-D and FIG. 7B show an embodiment of a surgical retractor 1600, 1700 that uses the LED flex circuit 1500b of FIGS. 5B-C. FIGS. 6A-D show the LED circuit 1500b assembly with a retractor blade cover 1606. FIG. 6A is an exploded view of the LED flex circuit 1500b and the retractor blade cover 1606, while FIG. 6B shows the positioning of the LED flex circuit 1500b relative to the blade cover 1606. As shown in these figures, the particular shape, size, and contour of the LED flex circuit 1500b is designed to correspond to a specific position on the retractor blade cover 1606 and to the openings formed in the retractor blade cover 1606. Specifically, the size and positioning of the LEDs and the wings 1604 or tabs and the thickness of the LED flex circuit are designed to smoothly fit with the retractor blade and the retractor blade cover 1606 so as not to impede with other functionalities of the retractor.

As shown in FIG. 6A, the LEDs 1602 are mounted on portions of the LED circuit 1500b substrate without wings 1604 and when the LED circuit 1500b is assembled with the blade cover 1606, the LEDs are positioned to align with the openings 1605 formed in the cover 1606 and the portions of the LED circuit on which the LEDs are mounted may be held by ribs or supporting projections 1605a formed on the inner surface of the blade cover 1606 at or near the edges of the openings 1605. As shown in FIGS. 6A-B, certain portions of the length of the LED flex circuit 1500b substrate which do not have LEDs mounted thereon include wings 1604 or tabs extending along the sides thereof. As described herein above, these wings 1604 increase the surface area of the LED flex circuit 1500b to which the copper layers are applied so as to increase thermal resistance.

A cross-sectional view of the blade cover 1606 with the LED circuit board 1500b along the line A-A in FIG. 6B is shown in FIG. 6C. In FIG. 6C, the LED 1602 of the LED circuit board 1500b is held within the opening 1605 in the blade cover 1606 and the ribs 1605a near the opening 1605 hold the LED circuit board portion on which the LED is mounted in place to prevent shifting of the LED. As also shown in FIG. 6C, the wings 1604 extend around the ribs 1605a so as to further improve retaining of the LEDs in place. With this construction, the LEDs are retained within and/or aligned with the openings 1605 formed in the blade cover 1606.

FIG. 6D shows another version of the blade cover 1606 assembled with the LED flex circuit 1500b which is modified to include a new method of securing the LED flex circuit 1500b to the blade cover 1606. In this version, the substrate of the LED flex circuit 1500b includes a plurality of slots 1609 and the blade cover 1606 includes a plurality of posts 1610 corresponding in positions to the plurality of slots 1609. In the illustrative embodiment of FIG. 6D, the slots 1609 are oval in shape and the posts 1610 have a cylindrical shape with a round or oval cross-section, and the widths of the slots 1609 are slightly smaller than the widths of the posts 1610 on the blade cover 1606. The slots 1609 are placed over the corresponding posts 1010 on the blade cover 1606, and when the posts 1610 are inserted into the corresponding slots 1609, the periphery of the slots 1609 grabs the posts 1610 due to the sizing of the slots 1609 and secures the LED flex circuit 1500b to the blade cover 1606. It is understood that the shape of the slots 1609 is not limited to oval shapes, and that the slots 1609 and the corresponding posts 1610 may have different shapes as long as the slots 1609 securely engage with the posts 1610. With this configuration, the need for narrow ridge retainers for holding the LEDs in place is eliminated, and the LED flex circuit can have an even larger surface area for the copper layers and the coverlays so as to dissipate more thermal energy and to lower the temperature of the LEDs. As can be seen in FIG. 6D, the area of the LED flex circuit 1500b substrate adjacent some of the LEDs 1602 has the same or similar width as that of the wings 1604, which provides the larger surface area.

When the LED flex circuit 1500b and the blade cover 1606 shown in FIGS. 6A-D are assembled with the blade portion of the retractor and the tubes of the smoke evacuation assembly, the wings 1604 of the LED circuit overlap with the tubes so as to hold the tubes 1612 in place relative to the blade portion. FIG. 7B shows an exploded view of the retractor that includes the LED flex circuit 1716 of this embodiment and the blade cover 1718 as well as the tubes of the smoke evacuation assembly. As shown in FIG. 7B, the tubes 1714 are inserted between a concave surface of the blade portion and the LED flex circuit 1716 assembled with the blade cover 1718, so that the tubes 1814 overlap with the wings of the LED flex circuit 1716 on each side thereof, and the blade cover 1718 is press-fit with the blade portion. Thus, the tubes are held in place between the wings of the LED flex circuit and the concave surface of the blade portion.

Referring back to FIGS. 6A-B and 6D, the blade cover 1606 includes a plurality of cylindrical posts or pins 1608. When the blade cover 1608 is press-fit with the blade portion, the pins 1608 on the blade cover are inserted into corresponding openings formed in the blade portion. In certain embodiments, the openings in the blade portion have a different shape, such as a hexagonal shape, and are sized so that when the pins 1608 of the blade cover are inserted into the openings, the pins 1608 have to deform and conform to the shape of the openings in the blade portion. Since the pins 1608 have a different shape from the openings in the blade portion, the pins may be made from a softer material so that the pins can conform to the shape of the openings. In some embodiments, the blade cover has pins with a first shape or cross-section, while the openings in the retractor blade have a second shape, different from the first shape. When the blade cover is press-fitted with the blade portion, the coupling strength between the blade and the cover is increased as well as the strength of the retractor blade is increased. With the blade cover strengthening the retractor blade, the retractors embodying such feature are able to withstand higher amounts of force and higher loads during use. Moreover, this construction of the blade and the blade cover 1608 allows for elimination of side flanges in the curved section connecting the blade portion to the handle portion, without sacrificing strength of the retractor.

Referring now to FIGS. 7A and 7B, these figures show exploded views of a retractor 1700 that incorporates the LED flex circuit 1500a of FIG. 5A or LED flex circuit 1500b of FIGS. 5B-5C described above. FIGS. 7C-7D show an internal view of the handle portion of the retractor in an assembled state. As shown in FIGS. 7A-B, the retractor 1700 includes the blade portion and the handle portion 1702, with the handle enclosing a push tab assembly. The push tab assembly includes a PCB assembly 1704, a power source, such as one or more batteries 1706, a battery door 1708, and a push tab 1710. The push tab assembly may also include, or may be electrically coupled to, an operating assembly 1712 for controlling the light sources (LEDs). In certain embodiments, the operating assembly includes a circuit, such as a flex circuit 1712a, for the controls, a button 1712b or another type of switch, and a wheel 1712c or a slide-type switch.

As shown in FIG. 7B, the retractor 1700 also includes a curved end cover 1720 which includes a suction or vacuum port and openings for accommodating the operating members 1712b, 1712c. The curved end cover 1720 may be attached to the curved portion of the handle using screws 1720a, 1720b, as shown in FIG. 7B, or using any other suitable fasteners or fastening assembly. For example, the curved end cover 1720 may be attached to the curved end of the handle portion by press-fitting posts or pins provided on one of the curved end cover and the curved end with corresponding openings provided on the other of the curved end cover and the curved end of the handle, which may have corresponding shapes or different shapes, similar to the press-fitting of the blade cover to the blade portion described herein above.

As shown in FIGS. 7A-B, the retractor 1700 further includes the pair of tubes 1714 which correspond to the tubes 1402 in FIGS. 4A-B, an LED flex circuit 1716 and the blade cover 1718. The LED flex circuit 1716 in FIG. 7A corresponds to the LED flex circuit 1404 in FIGS. 14A-B and LED flex circuit 1500a of FIG. 5A, and in FIG. 7B, corresponds to the modified LED flex circuit 1500b of FIGS. 5B-C and 6A-D. The blade cover 1718 of in FIGS. 7A-B corresponds to the blade cover 1406 in FIGS. 4C-D and to the blade cover 1606 in FIGS. 6A-D. As shown in FIG. 7B, the blade cover 1718 may be coupled to an end of the LED flex circuit 1716 and to the blade or the handle using a screw 1718a or using any other suitable fastener.

FIGS. 7C and 7D show the internal configuration of the handle portion of FIGS. 7A-B in an assembled state. As shown in FIGS. 7C and 7D, the PCB assembly 1704 is first positioned within the handle portion 1702 and thereafter the tubes 1714 are placed on top of the PCB assembly so that the tubes 1714 overlap with the sides of the PCB assembly 1704. The internal surface of the handle portion has ribs or projections 1702a projecting therefrom. The ribs 1702a are used for positioning the PCB assembly within the handle portion 1702 and for securing the tubes 1714 over the PCB assembly 1704. As shown in FIG. 7C, the tubes 1714 are pressed between the ribs, which hold the tubes in place and secure the PCB assembly 1704 in place within the handle portion.

FIG. 7D shows the internal configuration of the handle's curved end in an assembled state. As shown in FIG. 7D, the ends 1714a of the tubes 1714 are inserted and pinched by internal ribs 1702b formed within the handle. The curved end cover has similar internal ribs formed thereon which correspond to the internal ribs 1702b and which interact or engage with the internal ribs 1702b and also pinch the ends 1714a of the tubes 1714 so as to form a sealed chamber 1722 within the handle's curved end. In this way, smoke, fluids and/or debris conveyed from the operating site via the tubes empty out into the sealed chamber 1722 and are sucked out from the retractor via a suction or vacuum port (not shown). The internal construction of the sealed chamber 1722 is described in more detail in U.S. Pat. No. 10,512,519 incorporated herein by reference, and thus, detailed description thereof is omitted.

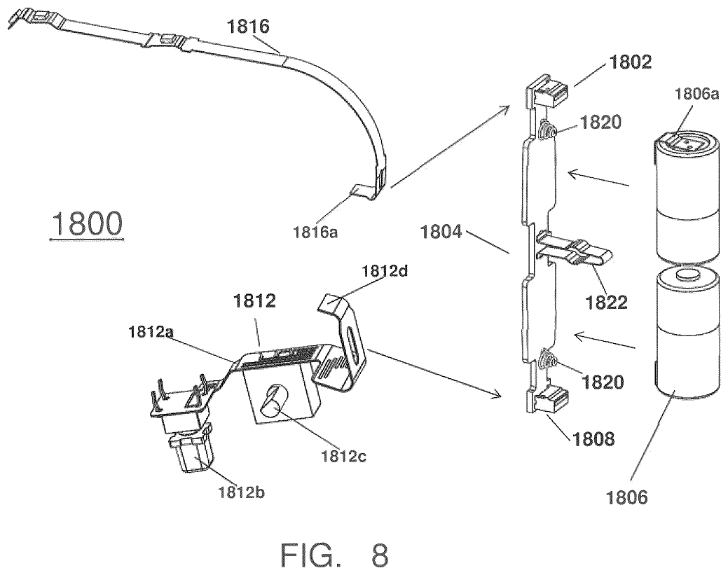

The details of the LED flex circuit 1716, as well as the power connections among the LED flex circuit 1716, the PCB assembly 1704, the batteries 1706, and the operating assembly 1712 will now be described with reference to FIG. 8. Although FIG. 8 shows the LED flex circuit 1404 of FIGS. 4A-B, it is understood that the modified LED flex circuit 1500b of FIGS. 5B-C and 6A-D may be used instead. The LED flex circuit 1500b of FIGS. 5B-C and 6A-D has similar electrical connections, e.g., exposed conductive traces, to be connected with the PCB assembly.

The LED flex circuit, as used in the retractor assemblies described herein, is a custom-fabricated circuit that is used to connect LEDs mounted thereon to a control PCB, i.e., PCB assembly. As described above, the function of the LED flex circuit is to allow for the use of surface-mounted LED packages on a circuit board substrate and to eliminate all single-conductor wires, thus improving ease of assembly.

As shown in FIG. 8, a rear end (proximal end) of the LED flex circuit 1816 has bare exposed conductive traces 1816a that are inserted into a first flex circuit connector 1802 mounted on the PCB assembly 1804. As described above, the distal end of the LED flex circuit 1816 includes one or more LEDs mounted thereon for illumination. To power the LEDs on the LED flex circuit 1816, one or more batteries 1806 are connected to the PCB assembly 1804 via springs 1820 or similar electrical contacts and a central contact 1822. The springs 1820 hold the batteries 1806 in place against a cover 1708 shown in FIG. 7, and electrically connect to the batteries 1806 via electrical connection plates 1806a, each of which is coupled to a battery terminal, or via similar electrical connections. The central contact 1822 provides electrical conductivity between the batteries. The springs 1820 and the central contact 1822 are also mounted on the PCB assembly 1804. It is to be noted that the particular size and shape of the central contact 1822 as shown in these figures are not intended to be limiting. Various other designs of the central contact may be incorporated without departing from the scope of the present invention.

The PCB assembly 1804 modulates power to the LEDs based on a number of different modes and brightness/color settings. These settings are controlled via the operating assembly 1812, which is also connected to the PCB assembly 1804 via a second flex circuit connector 1808. The second circuit connector 1808 is also mounted on the PCB assembly 1804. As shown in FIG. 8, the operating assembly 1812 includes a flex circuit 1812a with operating members mounted thereon, and the operating members can be operated by a user to change the mode and brightness/color settings. The flex circuit 1812a has a connecting portion 1812d which is adapted to connect with the second flex circuit connector 1808. In FIG. 8, the operating members include a button 1812b and a potentiometer 1812c, and when assembled in the handle portion, an outer portion of the button 1812b and an outer portion (not shown in FIG. 8) of the potentiometer 1812c, e.g., a slide or a rotary switch, are exposed through the handle portion to allow operation by a user. The assembled view of the handle portion with the button and the potentiometer can be seen, for example, in FIG. 3.

When each of the above components are assembled, the LED flex circuit 1816 is positioned mostly along the blade portion of the retractor, the PCB assembly is positioned mostly within the handle portion of the retractor, and the operating assembly 1812 is positioned mostly within the curved end portion of the retractor. However, these configurations are also not intended to limit the scope of the present invention and they may vary among various versions and variations without departing from the scope and spirit of the present invention. For example, in some versions, the operating assembly 1812 may be provided in other portions of the handle, e.g., near the proximal end of the handle.

Illuminated Retractors and Other Medical Devices with Magnet-Safe Shielding

As mentioned herein above, the retractors and other medical and surgical devices described herein are configured to be magnet-safe so as to be safely usable with magnetic and magnetic resonance (MR) equipment and within areas and localities that are exposed to externally generated magnetic fields and/or magnetic signals. Specifically, the retractors and other medical/surgical devices of the present embodiment include selectively applied shielding (shielding material) to predetermined portions of the device in preselected areas that are likely to be exposed to magnetic fields applied to a patient's body and that are likely to interfere with magnetic fields and signals generated by external equipment without such shielding. The shielding prevents the predetermined portions to which it is applied and/or internal components within or enclosed by those predetermined portions from interfering with and disrupting magnetic and electromagnetic fields and signals. In some cases, the predetermined portions are made with ferromagnetic materials, such as iron, cobalt and nickel, while in some cases, internal components of the device within the predetermined portions are made with ferromagnetic materials, while the predetermined portions themselves may or may not include ferromagnetic materials. In some embodiments, the predetermined portions in the preselected areas include the shielding material, while other portions of the device do not have any shielding material applied thereto. This limits electric conductivity of the device's outer housing and retains its radiolucent characteristics. However, in some cases, shielding material may be applied to portions of the device other than the predetermined portions or to the entire outer housing of the device. This may be the case if, for example, shielding material is applied for another purpose, such as for heat sinking, or where selective application of the shielding material is difficult, such as due to the size or shape of the predetermined portions.

Shielding material applied to the predetermined portions of the device includes one or more of paramagnetic materials, and especially low paramagnetic materials, diamagnetic materials and certain non-magnetic materials capable of shielding against magnetic/electromagnetic field interference. In certain embodiments, shielding material includes one or more of (1) paramagnetic materials, such as one or more of aluminum, copper, molybdenum, platinum, tungsten, titanium, and alloys thereof, and (2) diamagnetic materials, such as one or more of copper, gold, silver, zinc, bismuth, and alloys thereof. In yet other embodiments, shielding material comprises electrically and magnetically non-conductive ceramic and/or plastic/polymer materials, including but not limited to polytetrafluoroethylene (PTFE), nylon, carbon graphite, ceramics, etc.

In certain embodiments, shielding material is externally applied to cover the exterior or interior surface of the one or more predetermined portions of the device. In some embodiments, the external application of the shielding material may be accomplished by application of a sheet, foil, coating, film, encapsulant, veneer, laminate, and/or other component or application of shielding material to an external surface of one or more predetermined portions. The attachment methods of the shielding material include, but are not limited to, use of adhesives, bonding, press-fitting, latching, clipping, use of retention component(s) (e.g., spring), use of screws or pins, coating, embedding, etc. The thickness of the shielding material applied to the external surface may vary depending on the specific component being covered with shielding material, and in some embodiments, the thickness is less than 1 mm and preferably less than 0.5 mm. In certain embodiments, the thickness of the shielding material applied to the surface is between 0.01 and 0.5 mm, and preferably 0.01-0.1 mm or 0.05-0.1 mm, while in other embodiments, the shielding material thickness is substantially smaller, in the range of about 0.1-5 microns, preferably 0.1-3 microns.

In some embodiments, the shielding material is applied by electroplating it on the predetermined surfaces of the device. Specifically, the shielding material is applied by electroplating on interior surfaces and/or exterior surfaces of one or more predetermined portions of an outer housing of the device. In these embodiments, the thickness of the shielding material applied to the surfaces of the one or more predetermined portions of the outer housing is at least 0.001 mm, and in some embodiments, in between 0.001 mm and 0.51 mm.

Since the outer housing of some devices is formed from polymers or plastic, the predetermined surfaces of the outer housing to be coated have to be prepared and pretreated for electroplating to be carried out. In such cases, electroplating preparation is performed using electroless, auto-catalytic, chemical plating or using painting or coating of the predetermined surfaces using conductive paint prior to performing electroplating. These methods are described in more detail herein below in Examples 1 and 2.

Moreover, since the shielding material is applied to the predetermined portions of the outer housing of the device, and not to other portions of the outer housing, electroplating is selectively performed on the predetermined portions, while excluding other portions of the outer housing. Such selective electroplating can be performed by (1) applying masking material, either manually or automatically, to areas of the outer housing where electroplating is not required, or (2) fixture masking, wherein a masking fixture is temporarily attached to areas of the outer housing where electroplating is not required, or (3) using a two-shot injection molding technique, wherein portions of the outer housing that are not to be electroplated are injected with material that is resistant to etching solution, such a chromic acid etching solution, while the predetermined portions of the outer housing where electroplating is to be performed are injected with material that is susceptible to the etching solution. In certain embodiments, the selective electroplating is performed by coating or painting predetermined portions of the outer housing with conductive paint so that electroplating is performed in the areas where conductive paint is present.

In some embodiments, the shielding material is in a form of a paint or suspension and is applied to the external surface of one or more predetermined portions of the device by painting, brushing, dip coating, spin coating, flow coating, roll coating and/or spray coating, e.g., atomization, aerosol spray coating, thermal spray coating, plasma spray coating, etc. These types of shielding materials and applications thereof are particularly suitable where smaller thicknesses of the shielding material are desired. In certain embodiments, the shielding material thickness is 5 microns or less, preferably in the range of about 0.1-5 microns, and more preferably 0.1-3 microns. However, in other embodiments, the thickness of the shielding material applied to the surface is between 0.01 and 0.5 mm, and preferably 0.01-0.1 mm or 0.05-0.1 mm.

In the above examples, the shielding material may be applied to the external surface and/or the internal surface of one or more predetermined portions of the device so as to completely or partially coat/cover/encapsulate the external surface and/or the internal surface of the one or more predetermined portions. However, in certain embodiments, the shielding material may be applied to coat less than the entire external and/or internal surface of the one or more predetermined portions as long as the coating is sufficient to prevent interference with or disruption of magnetic and/or electromagnetic fields and signals.

In yet other embodiments, the shielding material is incorporated within the substrate material (main or core material) forming one or more predetermined portions of the medical device. For example, for predetermined portions formed from plastic or polymer materials, the plastic or polymer materials may be sprayed with shielding material(s) prior to use in injection molding. Alternatively, the shielding material(s) may be mixed with plastic or polymer materials prior to injection molding or plastic containing shielding material(s) may be prepared or selected prior to injection molding. In some embodiments, the substrate material forming one or more of the predetermined portions, including any plastic, polymer and/or metallic materials, is doped with one or more shielding materials, while in other embodiments, metallic main or core material forming one or more of the predetermined portions is alloyed with one or more shielding materials. The shielding material may also be embedded or free-floating within the one or more predetermined portions. Other suitable methods of incorporating the shielding material into the substrate material of the predetermined portion(s) of the medical device, or a combination of any of the above methods may also be used. In yet other embodiments, one or more of the predetermined portions may be formed from one or more shielding materials.

Although the above examples describe application of the shielding material to the external and/or internal surface of the predetermined portions of the device or incorporation of the shielding material within the material of the predetermined portions, it is understood that other methods of applying the shielding are suitable such that the shielding material is provided between the external magnetic or electromagnetic equipment and the portions of the device that contain ferromagnetic materials.

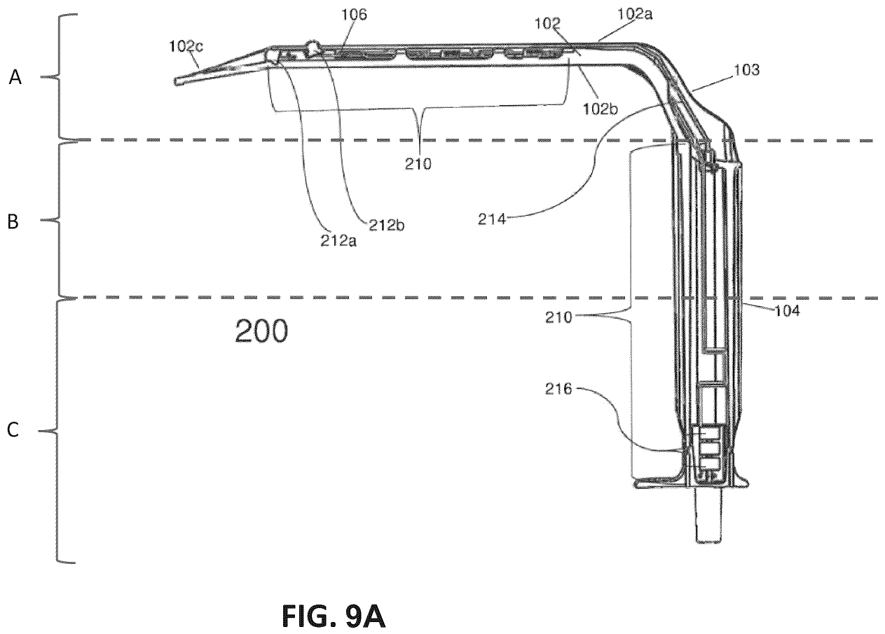

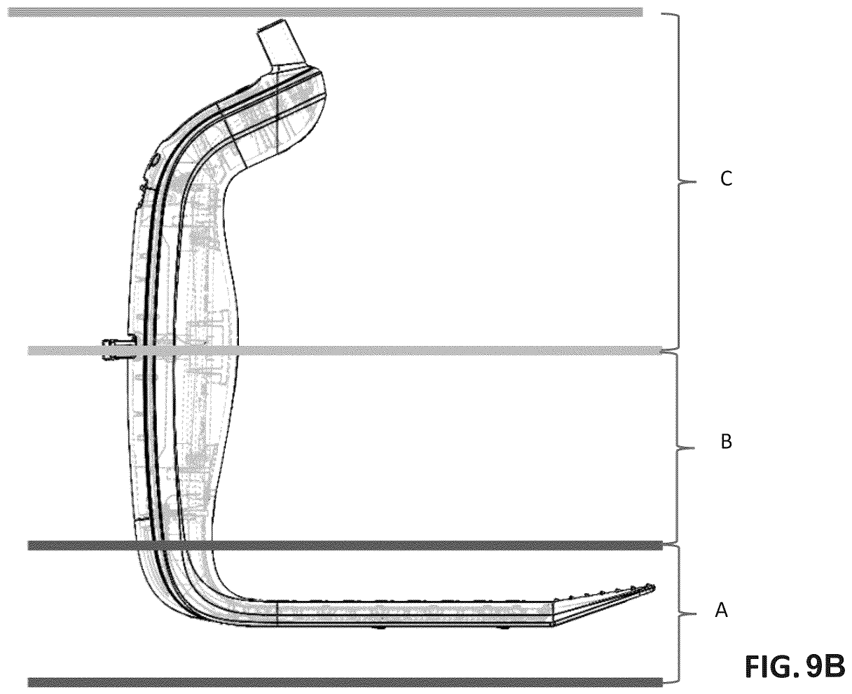

FIGS. 9A and 9B show the exemplary retractors of FIGS. 1-7B, respectively, with annotations to indicate portions of the respective retractor that are likely to interfere with external magnetic equipment. As shown in FIGS. 9A and 9B, the blade of the retractor and a portion of the handle adjacent to and attached to the blade is labeled as area “A”. This area “A” is likely to be within the surgical pocket but either does not include ferromagnetic materials or includes a limited number of components with ferromagnetic material that are likely to interfere with external magnetic/electromagnetic equipment.

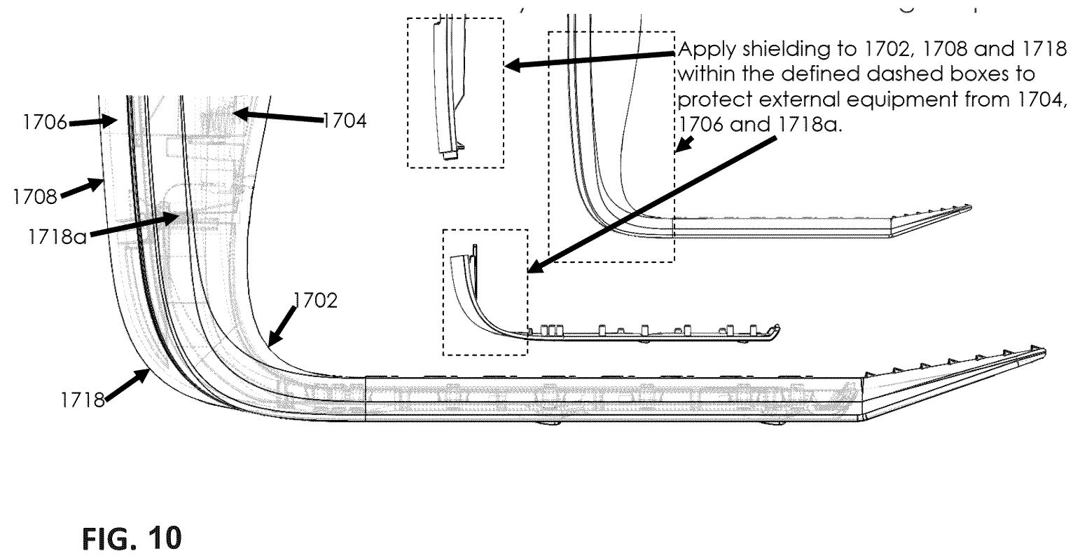

A portion of the handle labeled as “B” in FIGS. 9A-9B is an area that may be near the surgical pocket during use and contains ferromagnetic materials that are likely to interfere with external magnetic/electromagnetic equipment. As discussed with respect to FIGS. 9B and 10 and with respect to other devices, an area that may be near a surgical pocket during use includes an area of the device that would be positioned within 0-4 inches of a surgical pocket opening, or within 0-3 inches of a surgical pocket opening, or within 0-2 inches of a surgical pocket opening, or within 0-1 inches of a surgical pocket opening during typical use of the device. Therefore, portion “B” of the retractor includes one or more predetermined portions that require shielding material to be applied thereto to prevent interference with magnetic/electromagnetic equipment.

Finally, a third portion of the retractor which includes the distal end portion of the handle is labeled as “C” in FIGS. 9A-9B. This portion “C” is located further from the surgical pocket during use of the retractor and contains ferromagnetic materials. However, due to its location, the ferromagnetic materials in this portion are less likely to interfere with or disrupt the magnetic/electromagnetic equipment. Therefore, application of shielding material to portions containing ferromagnetic materials in this portion “C” is optional and is not required.

FIG. 10 shows enlarged views of portions of the retractor shown in FIG. 9B that are likely to be within or near a surgical pocket during use of the retractor In FIG. 10, the enlarged portion of the retractor includes a portion of the handle 1702, a portion of the battery door 1708 and a portion of the blade with the blade cover 1718 that are likely to be within or near the surgical pocket during use and which are likely to interfere with magnetic and electromagnetic fields and signals, i.e., which include or house ferromagnetic materials. In the illustrative example of FIG. 10, one or more shielding materials discussed above are applied to a portion of the handle 1702, a portion of the battery door 1708 and a portion of the blade with the blade cover 1718 within at least the areas surrounded by dashed lines, i.e., predetermined portions, which include a proximal end portion of the handle 1702, a proximal end portion of the blade and the blade cover 1718 and a portion of the battery door 1708 which is positioned at or near the curved joint between the handle 1702 and the blade.

In this illustrative example, the predetermined portions of the retractor to which the shielding material(s) are applied are select exterior and/or interior surfaces of the retractor's outer housing which include the predetermined portions of the handle 1702, the battery door 1708 and the blade with the blade cover 1718. The predetermined portions of the handle 1702, the battery door 1708 and the blade with the blade cover 1718 include: (1) the curved joint formed between the handle and the blade, (2) the proximal end portion of the blade with the blade cover 1718 that extends at least 1 inch from the curved joint in one direction, and (3) the proximal end portion of the handle 1702 that extends at least 1 inch from the curved joint in the other direction. In some embodiments, the proximal end portion of the handle 1702 to which the shielding material(s) are applied extends 1-2 inches from the curved joint, or 1-3 inches from the curved joint, or 1-4 inches from the curved joint so as to provide additional protection against interference with magnetic and electromagnetic signals. In other embodiments, the shielding material(s) are applied to at least ⅓ of the length of the handle 1702 from the curved joint, and in yet other embodiments, the shielding material(s) are applied to at least ½ of the length of the handle 1702 from the curved joint. Similarly, in some embodiments, the proximal end portion of the blade with the blade cover 1718 to which the shielding material(s) are applied extends 1-2 inches from the curved joint, or 1-3 inches from the curved joint, or 1-4 inches from the curved joint. In some embodiments, a majority portion of the blade with the blade cover 1718 or the entire blade with the blade cover 1718 may have shielding material(s) applied thereto, but such application is typically not required when copper or other similar coating is provided on the flexible LED circuit for heat sinking purposes and acts as a shielding material.

In certain embodiments, the shielding material(s) are applied to these predetermined portions of the handle 1702, the blade with the blade cover 1718 and the battery door 1708 forming the exterior surface of the body portion (i.e., outer housing) of the retractor so as to form a shielding layer of shielding material between ferromagnetic components housed inside the body portion of the retractor. In the present embodiment shown in FIG. 10, the ferromagnetic components housed inside the predetermined portions of the handle 1702, the blade with the blade cover 1718 and the battery door 1708 include the LED driver PCB of the PCB assembly 1704, the battery 1706 and the screw 1718a for the blade cover 1718. In such embodiments, the shielding material(s) may be applied to an interior surface of one or more of these predetermined portions of the handle 1702, the blade with the blade cover 1718 and the battery door 1708, or to an exterior surface of one or more of these predetermined portions, or to both the interior and exterior surfaces using any of the above application methods described above. In some embodiments, the shielding material(s) may be incorporated into the materials used for forming the handle 1702, the blade with the blade cover 1718 and/or the battery door 1708 using any of the methods described above.

In yet other embodiments, the shielding material(s) may be applied to or incorporated into specific components of the retractor that include ferromagnetic materials and that are housed in the handle, the blade and the curved joint regions of the retractor using the methods described above. This application/incorporation of the shielding material(s) may be in addition to or instead of the application of the shielding materials to the exterior body portions of the retractor, i.e., the handle 1702, the blade with the blade cover 1718 and the battery door 1708. For example, the shielding material(s) may be applied to or incorporated into the PCB assembly 1704, the battery 1706 and the screw 1718a for the blade cover, or portions thereof that are likely to interfere with magnetic and/or electromagnetic signals.

Referring to FIG. 7B which shows an exploded view of a retractor of the present invention, a table summary of an exemplary application of shielding material(s) to the retractor components is shown in Table 2500 below:

TABLE 2500

| | | Ferromagnetic/ | |
|---|---|---|---|
| | | Shielding | |
| ID | Description | Material | Comment |
| 1702 | Handle | — | Apply shielding (screw, |
| | | | battery, LED driver PCB) |
| 1704 | LED Driver | Ferromagnetic | To be shielded |
| | PCB | Material | |
| 1706 | Battery | Ferromagnetic | To be shielded |
| | | Material | |
| 1708 | Battery Cover | — | Apply shielding (screw, |
| | | | battery, LED driver PCB) |
| 1710 | Activator Tab | — | Shielding not necessary |
| 1712 | Control Board | — | Shielding not necessary |
| | subassembly | | |
| 1712a | Control fPCB | — | Shielding not necessary |
| 1712b | Button | — | Shielding not necessary |
| 1712c | Wheel | — | Shielding not necessary |
| 1714 | Silicone Tubing | — | Shielding not necessary |
| 1716 | LED fPCB | Shielding | Acts as a shield (copper |
| | | material | heat-sink) |
| 1718 | Blade Cover | — | Apply shielding (screw, |
| | | | battery, PCB) |
| 1718a | Screw | Ferromagnetic | To be shielded |
| | | Material | |
| 1720 | Control Cover | — | Shielding not necessary |
| 1720a | Screw | Ferromagnetic | Shielding not necessary; |
| | | Material | due to location |
| 1720b | Screw | Ferromagnetic | Shielding not necessary; |
| | | Material | due to location |

As shown in the above Table 2500, in the retractor of FIG. 7B, shielding material(s) are applied to the predetermined portions of the handle 1702, the battery cover 1708 and the blade with the blade cover 1718 in order to shield the LED driver PCB (PCB assembly 1704), the battery 1706 and the screw 1718a for the blade cover 1718. In certain embodiments, the shielding material(s) are applied to the inner surface of the predetermined portions of the handle 1702, the battery cover 1708 and the blade cover 1718 described above, wherein these predetermined portions are located in the area of the curved joint between the blade and the handle and in the proximal area of the handle. In other embodiments, the shielding material(s) may be applied to the outer surface of these predetermined portions of the handle 1702, the battery cover 1708 and the blade cover 1718 or to both the inner and outer surfaces of these predetermined portions. The application may be performed using any of the above-described methods.

In addition, as shown in Table 2500, the LED flex circuit 1716 (LED fPCB) is already formed from, or includes, shielding material with the copper heat-sink acting as a shield. Furthermore, shielding is not necessary, or is optional, for the push tab (activator tab) 1710, the operating assembly (control board subassembly) 1712, a flex circuit (control fPCB) 1712a, the button 1712b, the wheel 1712c, the tubes (silicone tubing) 1714 for smoke evacuation, the curved end cover (control cover) 1720, and the screws 1720a, 1720b for the control cover.

With reference to FIGS. 4A-4B, Table 2600 below summarizes application of shielding material(s) to portions of the retractor.

TABLE 2600

| | | Ferromagnetic/ | |
|---|---|---|---|
| | | Shielding | |
| ID | Description | Material | Comment |
| 1400 | Handle/blade portions | — | Apply shielding (screw, |
| | | | battery, PCB) |
| 1402 | Silicone Tubing | — | Shielding not necessary |
| 1404 | LED fPCB | Shielding | Acts as a shield (copper |
| | | material | heat-sink) |

As shown in Table 2600, shielding material(s) are applied to the predetermined portions of the handle and blade of the retractor 1400, particularly in the proximal end areas near the curved joint as described above with respect to FIG. 10, in order to shield the screw 1718a, the battery 1706 and the PCB assembly 1704. However, shielding is not necessary for the tubes 1402 (silicone tubing), which does not include ferromagnetic materials, or for the LED Flex circuit 1404 (LED fPCB) since the LED Flex circuit already includes copper coating to provide heat sinking which acts as a shield.

It is understood that the above summaries in Tables 2500 and 2600 are applicable to the specific retractor of FIGS. 7B and 4A-B and that other configurations of retractors with different components may need additional shielding or less shielding in the areas where ferromagnetic materials are likely to cause interference with magnetic and electromagnetic signals. For example, in the retractor shown in FIGS. 1A-1C and FIG. 9A, the shielding materials may be applied to the entire inner and/or exterior surfaces of the blade and blade cover, or to portions of the inner and/or exterior surfaces of the blade with illumination assembly components (e.g., light sources, wiring, etc.) adjacent thereto or housed therein. In other embodiments of the retractor of FIGS. 1A-1C and FIG. 9A, illumination assembly components (e.g., light sources, wiring, etc.), or metallic portions thereof, that are positioned adjacent to the blade may be made from the shielding materials, such as copper, or may have shielding material applied thereto, instead of or in addition to the shielding material being applied to the blade and blade cover.

Moreover, shielding material(s) may be similarly applied to other retractors (such as dual blade retractors described in applicant's U.S. Pat. No. 10,966,702, incorporated herein by reference and its related applications, nasal retractors shown in applicant's U.S. Pat. No. D904,607, incorporated herein by reference, other surgical retractors described in U.S. Pat. Nos. 10,966,699, 11,439,379 and 9,867,602, incorporated herein by reference and their related applications, orthopedic retractors, etc.). Furthermore, shielding material(s) may be applied to predetermined portions of other medical devices, including but not limited to, suction devices (such as the suction devices described in applicant's U.S. Pat. No. 10,959,609 and its related applications, all of which are incorporated herein by reference, and applicant's U.S. Pat. No. 10,722,621 and its related applications, all of which are incorporated herein by reference), electrocautery devices, laryngoscopes, anoscopes, speculum devices, cannulas and other types of surgical devices. The application of the shielding material(s) and the predetermined portions to which the shielding material(s) are applied will vary depending on the device components, and in certain embodiments, the application of the shielding material(s) is limited to the predetermined portions that contain ferromagnetic materials or that house components with ferromagnetic materials in the specific regions or areas where the device is likely to be within or near a surgical pocket and/or is likely to interfere with magnetic and electromagnetic signals. As discussed with respect to the retractor of FIGS. 9B and 10, the shielding material(s) are applied to the external and/or internal surfaces of predetermined portions of the retractor housing which house therein ferromagnetic components and which are likely to be within or near a surgical pocket during use. As also discussed above with respect to Tables 2500 and 2600, the shielding material(s) are not applied to portions of the retractor housing which house ferromagnetic components that already include materials, e.g., heat sinking materials, providing sufficient shielding on their own but applied thereto for a different function.

Examples 1 and 2 described below provides a detailed description of an exemplary magnet-safe retractor and exemplary electroplating methods of applying shielding materials to make the retractor magnet-safe.

### Example 1

In this illustrative example, the magnet-safe retractor includes a magnet-safe shielding material applied to the predetermined portions of the retractor housing, which include the proximal end portions of the handle and the battery cover, the curved joint between the handle and the blade and the proximal end portion of the blade and the blade cover. These predetermined portions are shown in FIG. 10 and are positioned within the dashed rectangular areas shown in FIG. 10. In this example, retractor outer housing, i.e., outer body forming the handle, the battery cover, the blade, the blade cover and the curved joint, is formed from plastic or polymer materials, such as polyarylamide, ABS or other suitable polymers. The shielding material is applied to the inner surface of the predetermined portions of the retractor housing, i.e., inner cavity of the predetermined portions, using electroplating techniques mentioned above. In this illustrative example, the shielding material is copper. However, it is understood that other shielding materials, such as aluminum (e.g., aluminum foil, nickel, etc. may be used.

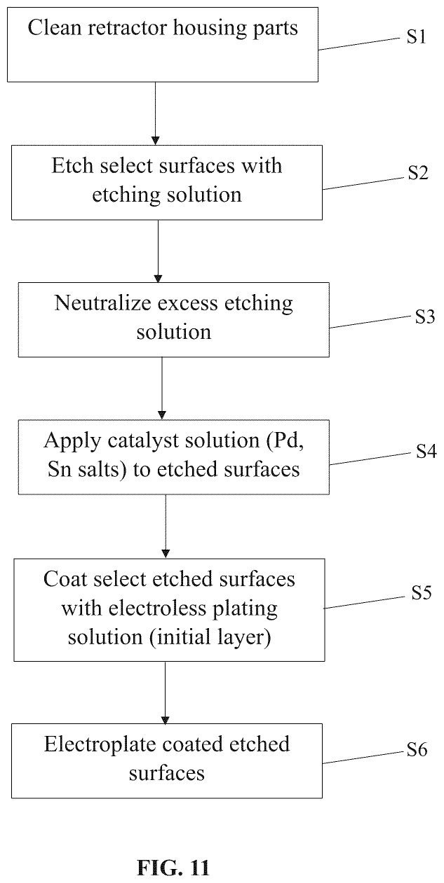

In this Example 1, the electroless, auto-catalytic, chemical plating process shown in FIG. 11 is used to electroplate the shielding material on the predetermined portions of the polymer outer housing. In this process, retractor housing parts to which the shielding material is to be applied are cleaned in step S1 so that these parts are free of all oil, grease, and foreign matter. The cleaning can be performed in any suitable way, including rinsing with acids, bases and other degreasing agents.

In step S2, select surfaces of the retractor housing parts to be electroplated are etched with an etching solution to enhance the adhesive capabilities of the select surfaces. In this example, chromic acid-based solution is used to etch the select surfaces. The select surfaces that are etched in step S2 correspond to the surfaces of the predetermined portions of the retractor housing and are the surfaces to which the shielding material is to be applied using electroplating.

As mentioned above, masking or injection molding techniques are used to selectively apply the etching to the select surfaces and to exclude other surfaces from being etched. Masking techniques include manual masking, wherein masking material is applied to areas of the housing parts where electroplating of shielding material is not to be applied. That is, masking material is applied to non-select surfaces of the retractor housing parts and is not applied to select surfaces of the retractor housing parts. This way, areas that are masked will not be etched with the etching solution, which will prevent the initial and subsequent layers of shielding material plating from adhering to these areas.

An alternative masking technique is fixture masking, wherein a fixture that conforms to the non-select surface of the housing part, i.e., surfaces where shielding material is not required, is temporarily attached to the housing part as a mask. This prevents the masked non-select surfaces, where the masking fixture contacts the surfaces, from being etched, and from the initial and subsequent layers of shielding material plating adhering to these surfaces.

Alternatively, two-shot molding techniques may be used instead of masking to selectively etch the select surfaces and to exclude the other surfaces from being etched. Certain types of plastics are inherently resistant to the chromic acid etching solution. Using the two-shot molding technique, portions of the housing part to which the shielding materials are not to be applied are injected with material that is resistant to chromic acid, while predetermined portions of the housing part to which the shielding materials are to be applied are injected with material that is susceptible to chromic acid. Exemplary polymer materials that are resistant to chromic acid include, but are not limited to: (i) Polypropylene, (ii) Polytetrafluoroethylene, (iii) Polyetheretherketone, (iv) Ethylene Chlorotrifluoroethylene, and (v) High-Density Polyethylene. Exemplary polymer materials that are susceptible to chromic acid include, but are not limited to: (i) Polyoxymethylene, (ii) Acrylonitrile Butadiene Styrene, (iii) Polycarbonate, and (iv) Poly(Methyl) Methacrylate. When two-shot molding techniques are used to form the predetermined portions and select surfaces of the housing parts from polymers that are susceptible to chromic acid, masking is not necessary prior to performing the etching in step S2 in order to selectively electroplate the select surfaces of the housing part.

In step S3, excess chromic acid etching solution is neutralized and in step S4, a catalyst solution is applied to the etched select surfaces of the housing parts. Specifically, in step S4, a solution of palladium and tin salts is applied to the etched select surfaces of the housing parts, and this solution acts as a catalyst when combined with copper or nickel shielding materials. the catalyst solution is selected specifically for the shielding material being applied, and may vary when different shielding materials are used.

In the next step S5, the select etched surfaces of the housing parts are coated with copper via the electroless plating solution. This coating results in an application of an initial layer of shielding material on the select surfaces of the housing parts, and the housing part is now ready for electroplating using standard plating technology.

In step S6, standard electroplating is performed to electroplate the coated etched surfaces of the housing part with the shielding material. In the present illustrative embodiment, the shielding material is copper and in step S6, the coated etched surfaces of the housing parts are electroplated with copper layers. In other embodiments, other shielding materials, e.g., aluminum, etc., may be used instead of copper.

As mentioned above, the minimum thickness of the resulting electroplated shielding material (e.g., copper) layer is 0.001 mm. The thickness of the electroplated shielding material is in the range of 0.001-0.51 mm.

### Example 2

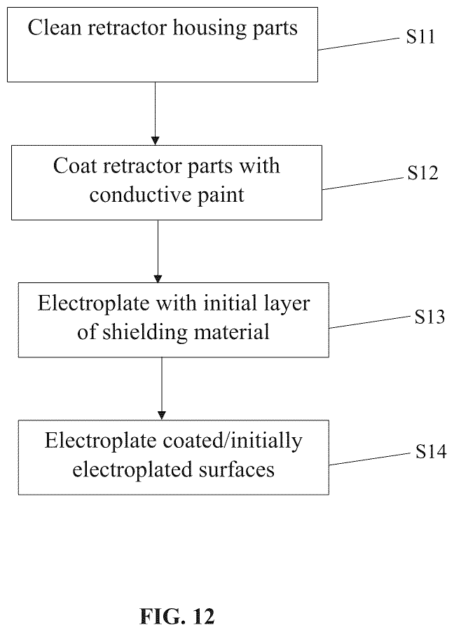

In this Example 2, the magnet safe retractor having a similar configuration to the retractor of Example 1 is prepared using a different process in which select surfaces of the housing parts of the retractor are coated using conductive paint before electroplating the select surfaces of the retractor housing parts. FIG. 12 shows this electroplating process.

As shown in FIG. 12, in step S11, retractor housing parts to which the shielding material is to be applied are cleaned so that these parts are free of all oil, grease and foreign matter. The cleaning can be performed in any suitable way, including rinsing with acids, bases and other degreasing agents.

In step S12, select surfaces of the retractor housing parts corresponding to the predetermined portions of the housing parts are coated with conductive paint. Although in this example, the surfaces that are coated with conductive paint correspond to the predetermined portions of the housing parts, while other parts are not coated, in other embodiments, the whole surface of the housing parts may be coated with the conductive paint.

In step S13, an initial layer of shielding material, such as copper, is electroplated on the painted surfaces of the housing parts. In the present example, since the conductive paint is present on the select surfaces of the housing parts, the copper shielding material will be electroplated on these select surfaces. After step S13, the housing parts are ready for electroplating using standard plating technology.

In the present example, the select surfaces of the housing parts are electroplated with copper layers in step S14 using standard plating technology. In other embodiments, other shielding materials, e.g., aluminum, etc., may be used. As mentioned above, the minimum thickness of the resulting electroplated shielding material (e.g., copper) layer is 0.001 mm. As mentioned above, the thickness of the electroplated shielding material is 0.001-0.51 mm.

### OTHER EMBODIMENTS

In accordance with the various embodiments that have been described herein, the retractors of the present application are made safe to use in magnetic and electromagnetic fields by applying shielding material(s) to predetermined portions of the retractor. The application of the shielding material(s) may also be performed on other medical and surgical devices. When the shielding material is applied to such other medical and surgical devices, the predetermined portions to which the shielding material(s) are applied are selected based on the location of ferromagnetic components within the device and whether the corresponding portion of the device will be inside or near a surgical pocket when the device is in use. As discussed in the exemplary embodiments with respect to surgical retractors, select interior or exterior surfaces of outer housings of these medical and surgical devices may be coated or plated with shielding material(s). Alternatively, or in addition to coating the housing surfaces, specific ferromagnetic components may be coated or plated with shielding material(s). As also discussed above, shielding material(s) may be incorporated into, or dispersed within, the materials forming the ferromagnetic components or the housing materials in order to make them magnet safe.

While various features and variations thereof have been described with respect to retractors, it is noted that one or more of the features described herein may be embodied within other medical devices which include ferromagnetic materials, such as a power source for use in an illumination assembly or for use in any other electrical assembly, including but not limited to speculums, anoscopes, laryngoscopes, suction devices, cannula and others.

In all cases, it is understood that the above-described arrangements are merely illustrative of the many possible specific embodiments which represent applications of the present invention. Numerous and varied other arrangements, including use of different materials and various configurations of components of the retractor, can be readily devised without departing from the spirit and scope of the invention.

We claim:

1. An illuminated surgical retractor comprising:
an outer housing forming a handle and a blade extending from the handle;
an illumination assembly comprising at least one direct light source,
wherein the outer housing houses therein one or more ferromagnetic components, and
wherein the outer housing includes an exterior surface and an inner surface and wherein predetermined portions of one or more of the exterior surface and the inner surface of the outer housing have a shielding material applied thereto to prevent interference of the one or more ferromagnetic components housed within the outer housing with magnetic and electromagnetic signals.

2. The illuminated surgical retractor in accordance with claim 1, wherein the shielding material is one or more of paramagnetic material, diamagnetic material and non-magnetic material capable of shielding against magnetic and electromagnetic field interference.

3. The illuminated surgical retractor in accordance with claim 1, wherein the shielding material is one or more of copper and aluminum.

4. The illuminated surgical retractor in accordance with claim 1, wherein the shielding material has a minimum thickness of 0.001 mm.

5. The illuminated surgical retractor in accordance with claim 1, wherein the predetermined portions of the one or more of the exterior surface and the inner surface of the outer housing include a joint between the handle and the blade, a proximal portion of the blade and a proximal portion of the handle.

6. The illuminated surgical retractor in accordance with claim 5, wherein the proximal portion of the blade extends at least 1 inch from the joint between the handle and the blade, and the proximal portion of the handle extends at least 1 inch from the joint between the handle and the blade.

7. The illuminated surgical retractor in accordance with claim 1, wherein the outer housing comprises a polymer material and wherein the shielding material is applied to the predetermined portions of the one or more of the exterior surface and the inner surface of the outer housing using electroplating.

8. The illuminated surgical retractor in accordance with claim 1, wherein:
the illumination assembly comprises a flexible circuit and the at least one direct light source is mounted on the flexible circuit,
the flexible circuit is housed within the blade and includes a flexible circuit board and copper layer provided on a top and a bottom surface of the flexible circuit board, and
the shielding material is not required to be applied to portions of the blade housing the flexible circuit board with the copper layer thereon.

9. The illuminated surgical retractor in accordance with claim 1, wherein the one or more ferromagnetic components include one or more ferromagnetic fastening members, one or more power sources housed in the handle and a control assembly housed in the handle for controlling power supply to the at least one direct light source.

10. The illuminated surgical retractor in accordance with claim 1, wherein at least one of the one or more ferromagnetic components includes shielding material directly applied thereto.

11. A medical device comprising:
an outer housing forming a handle and an operative portion extending from the handle;
one or more ferromagnetic components housed within the outer housing,
wherein the outer housing includes an exterior surface and an inner surface, and
wherein predetermined portions of one or more of the exterior surface and the inner surface of the outer housing have a shielding material applied thereto to prevent interference of the one or more ferromagnetic components housed within the outer housing with magnetic and electromagnetic signals.

12. The medical device in accordance with claim 11, wherein the shielding material is one or more of paramagnetic material, diamagnetic material and non-magnetic material capable of shielding against magnetic and electromagnetic field interference.

13. The medical device in accordance with claim 11, wherein the shielding material is one or more of copper and aluminum.

14. The medical device in accordance with claim 11, wherein the shielding material has a minimum thickness of 0.001 mm.

15. The medical device in accordance with claim 11, wherein the predetermined portions of the one or more of the exterior surface and the inner surface of the outer housing include portions of the outer housing enclosing the one or more ferromagnetic components and located inside a surgical pocket or within 0-4 inches of a surgical pocket opening when the medical device is in use.

16. The medical device in accordance with claim 15, wherein the predetermined portions of the one or more of the exterior surface and the inner surface of the outer housing include at least a portion of the operative portion, a proximal portion of the handle and a joint between the handle and the operative portion.

17. The medical device in accordance with claim 11, wherein the outer housing comprises a polymer material and wherein the shielding material is applied to the predetermined portions of the one or more of the exterior surface and the inner surface of the outer housing using electroplating.

18. The medical device in accordance with claim 11, wherein the one or more ferromagnetic components include one or more ferromagnetic fastening members and one or more power sources.

19. The medical device in accordance with claim 11, wherein at least one of the one or more ferromagnetic components includes shielding material applied thereto.

20. The medical device in accordance with claim 11, wherein the medical device is one of a retractor, a dual blade retractor, a suction device, a speculum, an anoscope, an electrocautery device and a laryngoscope.

21. An illuminated surgical retractor comprising:
an outer housing forming a handle and a blade extending from the handle;
an illumination assembly comprising at least one direct light source and one or more power sources,
wherein the outer housing is formed from a polymer material and houses therein one or more ferromagnetic components, and
wherein shielding material is selectively applied to one or more of predetermined portions of the outer housing and the one or more ferromagnetic components to prevent interference of the one or more ferromagnetic components with magnetic and electromagnetic signals.

22. The illuminated surgical retractor in accordance with claim 21, wherein the shielding material is one or more of copper and aluminum.

23. The illuminated surgical retractor in accordance with claim 21, wherein the shielding material has a minimum thickness of 0.001 mm.
